(19) United States
(12) Patent Application Publication
(10) Pub. No.: US 2020/0323922 A1
(43) Pub. Date: Oct. 15, 2020

(54) METHOD FOR TREATING A SIDE EFFECT OF IMMUNOTHERAPY

(71) Applicant: CYNATA THERAPEUTICS LIMITED, CARLTON, VICTORIA (AU)

(72) Inventors: KILIAN KELLY, CARLTON, VICTORIA (AU); IGOR SLUKVIN, VERONA, WI (US)

(73) Assignee: CYNATA THERAPEUTICS LIMITED, CARLTON, VICTORIA (AU)

(21) Appl. No.: 16/621,886

(22) PCT Filed: Jun. 13, 2018
(86) PCT No.: PCT/AU2018/050584

§ 371 Date: Dec. 12, 2019

(30) Foreign Application Priority Data
Jun. 16, 2017 (AU) 2017902301

(51) Int. Cl. A61K 35/28; A61P 35/02

(52) U.S. Cl. CPC A61K 35/28; A61P 35/02

(57) ABSTRACT

The invention relates to a method for treating a side effect of immunotherapy, the method comprising administering a mesenchymal stem cell (MSC) to a subject who has undergone or is undergoing immunotherapy. The invention also relates to a therapeutic composition comprising a MSC and a container comprising a MSC or therapeutic composition.

### FIELD

The invention relates to treating a side effect of immunotherapy.

### BACKGROUND

Immunotherapy is a biological therapy designed to improve a subject's native immune system to combat disease. Commonly, immunotherapy refers to cancer immunotherapy.

Established cancer immunotherapy includes cytokine therapy, whereas developing areas of cancer immunotherapy include checkpoint inhibitors, innate immune stimulators, and antibody conjugates.

Immunotherapy is demonstrating impressive responses in pre-clinical and clinical trials and the field is undergoing rapid expansion.

Despite its promise, immunotherapy is not without side effects and significant risk. Observed side effects include cytokine release syndrome (CRS) that may be related to macrophage activation syndrome (MAS), on-target, off-cancer effects leading to outcomes similar to graft-versus-host disease (GVHD) and B cell aplasia, tumour lysis syndrome (TLS), neurotoxicity such as cerebral oedema, and anaphylaxis caused by a subject's IgG response to non-human antigens.

CRS has been treated with standard supportive therapies, including steroids. However, steroids may affect T cells' activity or proliferation in the subject. Another therapy for CRS has been administration of inhibitors of pro-inflammatory cytokines that are elevated in CRS. Tocilizumab, an anti-interleukin 6 (IL-6) receptor antibody, and etanercept, a tumour necrosis factor (TNF) inhibitor, have been used to treat CRS.

B cell aplasia, resulting in reduced antibody production, has been treated with intravenous immunoglobulin to prevent infection.

TLS has been managed by standard supportive therapy, including hydration, diuresis, administration of allopurinol and recombinant urate oxidase, and haemodialysis as required.

Although these side effects have been managed with varying levels of success, they have not been entirely successful with adverse events occurring regularly, and even subject deaths occurring in a number of clinical trials.

Clearly an improved prophylactic and/or therapy for side effects of immunotherapy is required.

It is to be understood that if any prior art publication is referred to herein, such reference does not constitute an admission that the publication forms part of the common general knowledge in the art in Australia or any other country.

### SUMMARY

A first aspect provides a method for treating a side effect of immunotherapy, the method comprising administering a mesenchymal stem cell (MSC) to a subject who has undergone or is undergoing immunotherapy.

An alternative or additional embodiment of the first aspect provides use of a mesenchymal stem cell (MSC) in the manufacture of a medicament for treating a side effect of immunotherapy in a subject who has undergone or is undergoing immunotherapy.

A further alternative or additional embodiment of the first aspect provides a mesenchymal stem cell (MSC) for use in treating a side effect of immunotherapy in a subject who has undergone or is undergoing immunotherapy.

In one embodiment, the MSC has a CD73⁺CD105⁺CD90⁺CD146⁺CD44⁺CD10⁺CD31⁻CD45⁻ phenotype.

In one embodiment, the MSC expresses miR-145-5p, miR-181b-5p, and miR-214-3p, but not miR-127-3p and miR-299-5p.

In one embodiment, treating comprises administering to the subject about 1×10⁶ to about 1×10⁷ MSCs per kg body weight.

In one embodiment, treating comprises administering the MSC(s) within 24 hours after observing a side effect of immunotherapy.

In one embodiment, treating comprises administering the MSC(s) before, during or after immunotherapy. In one embodiment, treating comprises administering the MSC(s) after immunotherapy. In one embodiment, treating comprises administering the MSC(s) within 24 hours to 72 hours after immunotherapy.

In one embodiment, the side effect or symptom is: cytokine release syndrome (CRS), optionally release of IL-6, interferon-γ (IFN-γ), TNF, IL-2, IL-2-receptor α, IL-8, IL-10, or granulocyte macrophage colony-stimulating factor (GMCSF); macrophage activation syndrome (MAS); an on-target, off-cancer effect, optionally B cell aplasia; tumour lysis syndrome (TLS); neurotoxicity, optionally cerebral oedema; or anaphylaxis.

In one embodiment, the immunotherapy is for treating: a lymphoma; a leukaemia; a melanoma; an epithelial cancer; or a sarcoma.

In one embodiment, the immunotherapy is for treating: diffuse large B cell lymphoma (DLBCL); Hodgkin lymphoma; non-Hodgkin lymphoma (NHL); a non-Hodgkin B, T or NK cell lymphoma; primary mediastinal B cell lymphoma (PMBCL); transformed follicular lymphoma (TFL); mantle cell lymphoma (MCL); multiple myeloma (MM); chronic lymphocytic leukaemia (CLL); acute myeloid leukaemia (AML); or acute lymphoblastic leukaemia (ALL).

In one embodiment, the immunotherapy is a checkpoint inhibitor, a bispecific T cell engager, a stimulator of interferon genes agonist, a RIG I like receptor agonist, a Toll-like receptor agonist, a cytokine, an antibody-cytokine fusion protein, or an antibody-drug conjugate.

In one embodiment, the subject is mammalian, optionally human.

A second aspect provides a therapeutic composition for treating, ameliorating, or reducing a side effect of immunotherapy in a mammalian subject, wherein said therapeutic composition comprises a mesenchymal stem cell (MSC), wherein the MSC is made by a method comprising:

(a) culturing a primitive mesoderm cell in a mesenchymal-colony forming medium (M-CFM) comprising LiCl and FGF2, but excluding PDGF, under normoxic conditions for sufficient time for a mesenchymal colony to form; and

(b) culturing the mesenchymal colony of (a) adherently to produce the MSC,

wherein the MSC of (b) expresses miR-145-5p, miR-181b-5p, and miR-214-3p, but not miR-127-3p and miR-299-5p, and/or has phenotype CD73⁺CD105⁺CD90⁺CD146⁺CD44⁺CD10⁺CD31⁻CD45⁻.

A third aspect provides a container comprising a MSC that expresses miR-145-5p, miR-181b-5p, and miR-214-3p, but not miR-127-3p and miR-299-5p, and/or has phenotype CD73⁺CD105⁺CD90⁺CD146⁺CD44⁺CD10⁺CD31⁻CD45⁻.

A fourth aspect provides a container comprising the therapeutic composition of the second aspect.

### BRIEF DESCRIPTION OF THE FIGURES

FIG. 1 is a schematic representation of the experimental design of Example 14.

FIG. 2 is a graph showing the rectal temperature of control and test mice of Example 14.

FIG. 3 is a graph showing clinical score of control and test mice of Example 14. 0=Normal activity; 1=Normal activity, piloerection, tiptoe gait; 2=Hunched, reduced activity but still mobile; 3=Hypomotile, but mobile when prompted; 4=Moribund, euthanized.

FIG. 4 is a set of graphs showing percent mouse CD45+ cells, percent human CD45+ cells, CD4+ cells as a percent of human CD45+ cells, and CD8+ cells as a percent of human CD45+ cells in peripheral blood of mice of Example 14.

FIG. 5 is a set of graphs showing CD69 expression on human CD4+ T cells in peripheral blood of mice of Example 14.

FIG. 6 is a set of graphs showing CD69 expression on human CD8+ T cells in peripheral blood of mice of Example 14.

FIG. 7 is a set of graphs showing percent mouse CD45+ cells, percent human CD45+ cells, CD4+ cells as a percent of human CD45+ cells, and CD8+ cells as a percent of human CD45+ cells in spleen of mice of Example 14.

FIG. 8 is a set of graphs showing CD69 expression on human CD4+ T cells in spleen of mice of Example 14.

FIG. 9 is a set of graphs showing CD69 expression on human CD8+ T cells in spleen of mice of Example 14.

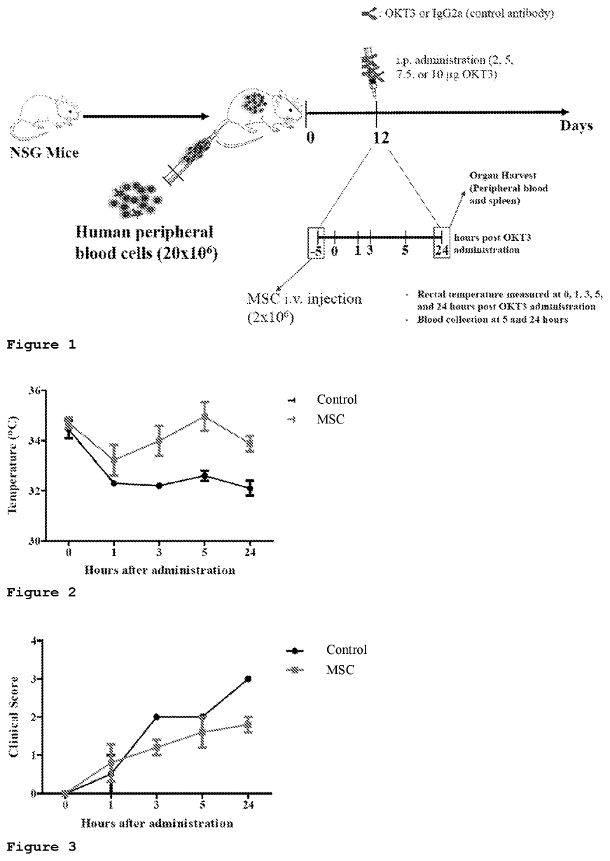

### DETAILED DESCRIPTION

Unless defined otherwise in this specification, technical and scientific terms used herein have the same meaning as commonly understood by the person skilled in the art to which this invention belongs and by reference to published texts.

It is to be noted that the term “a” or “an” refers to one or more, for example, “a MSC” is understood to represent one or more MSCs, including a population of MSCs. As such, the terms “a” or “an”, “one or more,” and “at least one” may be used interchangeably herein.

In the claims which follow and in the description of the invention, except where the context requires otherwise due to express language or necessary implication, the word “comprise” or variations such as “comprises” or “comprising” is used in an inclusive sense, i.e. to specify the presence of the stated features, but not to preclude the presence or addition of further features in various embodiments of the invention.

The term “about” as used herein contemplates a range of values for a given number of ±25% the magnitude of that number. In other embodiments, the term “about” contemplates a range of values for a given number of ±20%, ±15%, ±10%, ±5%, ±4%, ±3%, ±2%, or ±1% the magnitude of that number. For example, in one embodiment, “about 3 grams” indicates a value of 2.7 grams to 3.3 grams (i.e. 3 grams±10%), and the like.

Similarly, the timing or duration of events may be varied by at least 25%. For example, while a particular event may be disclosed in one embodiment as lasting one day, the event may last for more or less than one day. For example, “one day” may include a period of about 18 hours to about 30 hours. In other embodiments, periods of time may vary by ±20%, ±15%, ±10%, ±5%, ±4%, ±3%, ±2%, or ±1% of that period of time.

As used herein, “immunotherapy” includes, but is not limited to, treating a subject with: a checkpoint inhibitor; a bispecific T cell engager; a stimulator of interferon genes; a RIG I like receptor; a toll-like receptor; an antibody-cytokine fusion protein; a cytokine; or an antibody-drug conjugate.

Immunotherapies need not be used singly, and may be used in combination. For example, two checkpoint inhibitors may be combined, e.g. nivolumab and pembrolizumab or nivolumab and ipilimumab, or a checkpoint inhibitor may be combined with a conventional cancer therapy, e.g. radiotherapy or chemotherapy.

As used herein, “antibody” is used broadly and refers to an antigen binding molecule. Thus, the term “antibody” includes an immunoglobulin, such as IgA, IgD, IgE, IgG, IgM, IgY or IgW, a fragment, such as Fab, (Fab′)₂, scFv, scFv-Fc, a minibody, a diabody, a single domain antibody (sdAb or nanobody), a bispecific antibody, a multispecific antibody, and an antibody mimetic, such as an affibody, affilin, affimer, affitin, alphabody, anticalin, avimer DARPin, fynomer, Kunitz domain peptide and monobody. sdAbs include camelid antibodies and IgNARs from cartilaginous fish. An antibody may be polyclonal or monoclonal (mAb). An antibody may be chimeric, humanized or human.

Antibody production is well-known in the art and includes hybridoma, phage display, single B cell culture, and single B cell amplification technologies, for example. Chimeric and humanised antibodies may be produced using recombinant techniques. Human antibodies may be produced using phage display technology or transgenic animals such as transgenic mice, platforms for which are available commercially. Once an antibody with appropriate specificity has been identified and its sequence determined, the antibody may be produced recombinantly, for example in cell culture, for example CHO cell culture, as is known in the art.

As used herein a “side effect” includes a “symptom” and both terms refer to an undesired or adverse effect of immunotherapy, determined either qualitatively, i.e. undesired in any form, or quantitatively, undesired above or below a specific threshold. Such a symptom may also be referred to as an “adverse symptom” to distinguish an effect from a necessary or desired effect of immunotherapy. A side effect or symptom of immunotherapy may also be referred to as an “adverse event”, an “immune-mediated adverse event”, or an “immune-related adverse event”.

Checkpoint Inhibitors

Checkpoints are proteins that negatively regulate T cell immune responses. To date, two checkpoints have been identified: cytotoxic T lymphocyte antigen-4 (CTLA-4 or CD152) and programmed death-1 (PD-1 or CD279). PD-1 is activated by programmed death-ligand 1 (PD-L1) and programmed death-ligand 2 (PD-L2). Inhibition of checkpoints or their ligands abrogates the negative regulation of T cells and shifts the immune response toward T cell activation.

Ipilimumab is a human anti-CTLA-4 mAb. Ipilimumab may be administered at 3 mg/kg every 2 weeks or 3 weeks or at 10 mg/kg every 2 weeks, for example. Ipilimumab may be used to treat melanoma, non-small cell lung carcinoma (NSCLC), small cell lung cancer (SCLC), bladder cancer, and prostate cancer, for example, although clinical trials assessing ipilimumab for treating many more cancers are underway. Ipilimumab may be used in combination with other agents, for example nivolumab, bavituximab, dacarbazine, IL-2 or gp100.

Side effects or symptoms of ipilimumab include pruritus, rash, vitiligo, diarrhoea, colitis, increased ALT, increased AST, hepatitis, hypothyroidism, hypopituitarism, hypophysitis, adrenal insufficiency, increased thyrotropin, decreased corticotropin, acute inflammatory demyelinating polyneuropathy, ascending motor paralysis, and myasthenia gravis, for example.

Nivolumab is a human IgG4 anti-PD-1 mAb. Nivolumab may be administered at 3 mg/kg every 2 weeks, for example. Nivolumab may be used to treat melanoma, metastatic melanoma, metastatic squamous NSCLC, renal cell carcinoma, and bladder cancer, for example, although clinical trials assessing nivolumab for treating many more cancers are underway. Nivolumab may be used in combination with other agents, for example ipilimumab or pembrolizumab.

Side effects or symptoms of nivolumab include pruritus, rash, vitiligo, diarrhoea, colitis, increased ALT, increased AST, increased bilirubin, pneumonitis, increased serum creatinine, renal failure, hypothyroidism, hyperthyroidism, increased TSH, diabetes, hypophysitis, adrenal insufficiency, and fatigue, for example.

Pembrolizumab is a humanised anti-PD-1 mAb. Pembrolizumab may be administered at 2 mg/kg or 10 mg/kg every 2 weeks or 3 weeks, for example. Pembrolizumab may be used to treat melanoma, metastatic melanoma, metastatic NSCLC, head and neck squamous cell carcinoma (HNSCC), and Hodgkin's lymphoma, for example. Clinical trials assessing pembrolizumab for treating many more cancers, such as breast cancer, gastric cancer, and urothelial cancer, for example, are underway. Pembrolizumab may be used in combination with other agents, for example nivolumab, talimogene laherparepvec, dabrafenib plus trametinib, or ipilimumab.

Side effects or symptoms of pembrolizumab include pruritus, rash, rash maculopapular, dermatitis acneiform, diarrhoea, colitis, hepatitis, increased ALT, increased AST, dyspnea, pneumonitis, hypothyroidism, hyperthyroidism, increased amylase, pancreatitis, arthralgia, uveitis, pyrexia, and fatigue, for example.

Another anti-PD-1 antibody in development is BGB-A317, whereas anti-PD-L1 antibodies include atezolizumab, avelumab and durvalumab. Also in development is an anti-PD-L1 affimer.

Bispecific T Cell Engagers (BiTEs)

A bispecific T cell engager (BiTE) is a type of bispecific antibody that in a single protein links an scFv targeting a T cell surface antigen, for example CD3e, to an scFv targeting a surface antigen on a cancer cell. BiTEs are manufactured using recombinant techniques as known in the art.

The first in class BiTE is blinatumomab, which is a bispecific antibody directed to CD3e and CD19, and generally may be used to treat B cell malignancies, such as acute lymphoblastic leukaemia (ALL), non-Hodgkin's lymphoma (NHL), diffuse large B cell lymphoma (DLBCL), and chronic lymphocytic leukaemia (CLL), primary mediastinal B cell lymphoma (PMBCL), transformed follicular lymphoma (TFL), multiple myeloma (MM), mantle cell lymphoma (MCL), and acute myeloid leukaemia (AML). Blinatumomab may be administered at 5 μg/m²/d to 60 μg/m²/d, for example 5, 15 or 60 μg/m²/d.

The most common side effects of blinatumomab are CRS, pyrexia, fatigue, headache and weight change. CRS would be expected for most BiTEs.

Because BiTE is a platform technology, other cancer targets are possible, some of which include CD20, CD33, Epithelial cell adhesion molecule (EpCAM), carcinoembryonic antigen (CEA), prostate specific membrane antigen (PSMA), human epidermal growth factor receptor 2 (HER2), epidermal growth factor receptor (EGFR), Ephrin type-A receptor 2 (EphA2), mucin 1 (MUC1), and melanoma-associated chondroitin sulfate proteoglycan (MCSP).

Stimulator of Interferon Genes (STING)

Stimulator of interferon genes (STING, also known as TMEM173) is a signalling molecule that binds and is activated by cyclic dinucleotides, such as cyclic GMP-AMP and cyclic di-AMP, which are produced in response to DNA entering the cytosol. STING also binds double-stranded DNA. Upon activation, STING leads to IRF3-mediated and NF-κB-mediated transcription of type I interferons (IFNs) and inflammatory cytokines such as TNF, IL-1B, and IL-6, which in turn cause cell death and promotes dendritic cell, natural killer and CD8⁺ T cell function. Amongst other cells, STING is present in intratumoural dendritic cells.

Cyclic dinucleotides may be produced biosynthetically/enzymatically as is known in the art, although this is weighted towards naturally-occurring cyclic dinucleotides. Otherwise, cyclic dinucleotides may be produced chemically by nucleotide cyclization or by stereospecific base insertion on a cyclic bis(3′-5′)-sugar phosphate as is known in the art. Both solution-phase and solid-support syntheses have been developed and are known in the art.

Cyclic dinucleotides, including synthetic dithio mixed-linkage cyclic dinucleotides, for example, are under development as immunotherapy for cancer. Agents under development include ML RR-S2 CDA and MIW815, and SB 11285. Cyclic dinucleotides may be combined with other therapies such as radiotherapy, chemotherapy, checkpoint inhibitors, or lethally irradiated GM-CSF-secreting tumour cell cells (STINGVAX).

STING agonists may be used to treat melanoma and colon cancer, for example.

RIG I Like Receptors (RLRs)

RIG I like receptors include retinoic acid inducible gene I (RIG-I or DDX58), melanoma differentiation-associated protein 5 (MDA5 or IFIH1), and Laboratory of Genetics and Physiology 2 (LGP2 or DHX58), which are cytosolic RNA sensors.

RIG-I typically recognizes 5′-triphosphorylated RNA (5′-ppp-RNA or 3pRNA) and short double stranded RNA, and is dependent on functional LGP2. MDA5 typically recognises double stranded RNA longer than 2000 nucleotides, and is also dependent on functional LGP2. LGP2 itself cannot induce signalling, but is required for RIG-I-mediated and MDA5-mediated responses.

Upon activation, RIG-I and MDA5 lead to IRF1-, IRF3-, IRF7- and NFκB-mediated expression of IFNs and inflammatory cytokines, which that can directly act an tumour cells as well as activate T cells and natural killer cells.

RLR ligands may be used for immunotherapy and include 5′-ppp-siRNAs, which act in a sequence independent manner via the RLR pathway as well as in a sequence dependent manner via the RNA interference (RNAi) pathway, as well as the hemagglutinating virus of Japan envelope (HVJ-E) vector, and polyinosinic: polycytidylic acid (poly I:C).

RLR activation may be used to treat melanoma, pancreatic cancer, prostate cancer, glioma, malignant pleural mesothelioma (MPM), and ovarian cancer, for example.

Toll-Like Receptors (TLRs)

To date, ten human toll-like receptors (TLRs) have been identified, each with a different ligand specificity: TLR2 recognises lipoproteins and peptidoglycans; TLR3 recognises viral double stranded RNA and poly I:C; TLR4 recognises lipopolysaccharides (LPS); TLR5 recognises bacterial flagellin; TLR7/8 recognises single stranded RNA; TLR9 recognises CpG-containing oligodeoxynucleotides (CpG-ODN); and TLR2/6, TLR2, and TLR4 recognise the matrix proteoglycan versican and heat-shock proteins (HSPs).

A number of these ligands have been approved for cancer immunotherapy, including TLR2/4 agonists Bacillus Calmette-Guérin (BCG) for treating bladder cancer, TLR4 agonist monophosphoryl lipid A (MPL) for treating cervical cancer, and TLR7 agonist imiquimod for treating breast cancer. Coley toxin and extract of larix leptolepis (ELL) are also approved for immunotherapy.

Other TLR ligands are being developed for immunotherapy, for instance, TLR5 agonist flagellin-derived CBLB502 (Entolimod) for treating advanced solid tumours and hepatoma, TLR7 agonist 852A for treating melanoma and hematologic malignancy, and TLR3 agonist poly I:C/polyinosinic-polycytidylic acid stabilised with polylysine and carboxymethylcellulose (poly-ICLC) and TLR9 agonist synthetic CpG-ODN as cancer vaccine adjuvants for treating multiple cancer types including glioblastoma.

Thus, TLR ligands may be used to treat bladder cancer, breast cancer, cervical cancer, glioblastoma, hematologic malignancy, hepatoma, melanoma, and solid tumours, for example.

Cytokines

IL-2 is used clinically as an immunotherapy to improve the anticancer efficacy of cytotoxic T cells. IL-2 has also been used to promote ex vivo T cell expansion for adoptive cell transfer (ACT). Other cytokines in use or development for immunotherapy include IL-7, IL-12, IL-15, IL-18. IL-21. IFNα, IFNβ, IFNγ, granulocyte-macrophage colony stimulating factor (GM-CSF), and TNF. Cytokines may be produced recombinantly as known in the art.

Cytokines may be used to treat melanoma, renal cell carcinoma, lymphoma, B cell lymphoma, follicular lymphoma, hairy cell leukaemia, sarcoma, hepatitis B/C, chronic granulomatous disease, malignant osteoporosis, breast cancer, prostate cancer, sarcoma, Ewing's sarcoma, Kaposi's sarcoma, neuroblastoma, mycosis fungoides, head and neck cancer, AML, lung cancer, ovarian cancer, chronic myeloid leukaemia (CML), CLL, and neutropenia, for example.

Often, however, cytokines fail to reach a therapeutic concentration at the tumour site because they have no means for preferential trafficking and a very short initial serum half-life (minutes to hours). Also, cytokines have severe side effects including systemic inflammation and vascular leak syndrome. Other side effects include fever, chill, malaise, hypotension, organ dysfunction, and cytopenias.

Antibody-Cytokine Fusion Proteins

In view of the limitations of free cytokines in immunotherapy, improvements have been made by recombinantly fusing cytokines to antibodies of desired antigen specificity. Not only do such antibodies traffic cytokines to the tumour site of action, the antibodies may exert their own immunotherapeutic effects via antibody-dependent cellular cytotoxicity (ADCC) and complement-dependent cytotoxicity (CDC).

Antibody-cytokine fusion proteins include: hu14.18-IL-2, a humanised anti-GD2 IgG fused to IL-2, for treating neuroblastoma; DI-Leu16-IL2, a de-immunised and mutated anti-CD20 IgG fused to IL-2, for treating B cell malignancies; L19-IL2, a high affinity anti-fibronectin extradomain B diabody fused to IL-2, for treating lymphomas, metastatic melanoma, renal cell carcinoma and solid tumours; anti-CD20-IL-21, an anti-CD20 IgG fused to IL-21, for treating DLBCL and MCL; BC1-IL12, an anti-human fibronectin isoform B-FN (extradomain B) IgG, for treating malignant melanoma and RCC; L19-TNFα, an anti-fibronectin extradomain B human scFv fused to TNF, for treating melanoma, solid tumours, colorectal cancer; anti-CD20-IFNα, a chimeric anti-CD20 IgG fused to IFNα, for treating B cell lymphomas and leukaemias; anti-CD20-tetrameric hIFNα (20-2b-2b), humanised anti-CD20 IgG veltuzumab fused to four molecules of IFNα2b, for treating B cell lymphomas; F16-IL2, an anti-tenascin C extradomain A1 human scFv fused to IL-2, for treating breast cancer, lung cancer, Merkel cell carcinoma (MCC), glioblastoma, AML and solid tumours; NHS-IL2LT, a mouse-human chimeric antibody directed against DNA released by necrotic tumour cells fused to two molecules of IL-2, for treating solid tumours, NHL and NSCLC; NHS-IL12, a mouse-human chimeric antibody directed against DNA released by necrotic tumour cells fused to two molecules of IL-12, for treating renal cell carcinoma, Kaposi sarcoma, T cell lymphoma, and NHL; C2-2b-2b, a humanised anti-HLA-DR IgG fused to four molecules of IFNα2b, for treating haematopoietic cancers, B cell lymphomas and leukaemias, and MM; and huKS-IL2, an anti-epithelial cell adhesion molecule (EpCAM or KS) IgG fused to IL-2, for treating ovarian cancer, prostate cancer, colorectal cancer and NSCLC.

Side effects of hu14.18-IL-2 resembled those of IL-2 immunotherapy and included capillary leak, hypoxia, elevated transaminases, hyperbilirubinemia, hypotension, and renal insufficiency.

Antibody-Drug Conjugates (ADCs)

Anti-cancer drugs may be conjugated to antibodies in order to specifically target the drug to cancer cells. This approach is particularly useful in delivering anti-cancer drugs that exert cytotoxicity at concentrations much below standard chemotherapeutic drugs and thus are too toxic to administer in their free form. Chief amongst these are microtubule inhibitors such as maytansinoids and auristatins. Alternatively, the drug may be a radionuclide.

The antibodies of ADCs are commonly, but not necessarily, IgGs, often IgG1.

Drug, including radionuclide, production is known in the art.

The antibody and drug may be conjugated using a linker, which may be cleavable or non-cleavable. The linker may be cleavable once the ADC is internalised by a cancer cell, but stable in the circulation prior to internalisation. However, once cleaved, a drug may escape the targeted cell and attack non-cancer bystander cells. A non-cleavable linker is intended to retain the ADC within the cell.

Methods for conjugation are known in the art. Cleavable linker chemistry may employ disulfides, hydrazones or peptides, whereas non-cleavable linker chemistry may employ thioethers. Antibodies may be engineered to manipulate linker chemistry, for example cysteine amino acid residues may be manipulated to modify the number and/or position of sulfhydryl groups available for linker chemistry.

Two ADCs, brentuximab vedotin targeting CD30 and trastuzumab emtansine targeting HER2, are approved for clinical use, and many ADCs are in development, including inotuzumab ozogamicin (CD22), gemtuzumab ozogamicin (anti-CD33), ABT-414 (anti-EGFR), glembatumumab vedotin (anti-gpNMB), labetuzumab govitecan (anti-CEACAM5), sacituzumab govitecan (anti-TROP2, EGP1), lifastuzumab vedotin (anti-NaPi2b), indusatumab vedotin (anti-GCC), polatuzumab vedotin (anti-CD79b), pinatuzumab vedotin (anti-CD22), PSMA ADC (anti-PSMA), coltuximab ravtansine (anti-CD19), BMS-986148 (anti-MSLN), indatuximab ravtansine (anti-CD138, syndecan 1), milatuzumab doxorubicin (anti-CD74), MLN2704 (anti-PSMA), SAR408701 (anti-CEACAM5), rovalpituzumab tesirine (anti-DLL3), ABBV-399, AGS-16C3F (anti-ENPP3), ASG-22ME (anti-nectin 4), AGS67E (anti-CD37), AMG 172 (anti-CD27), AMG 595 (anti-EGFRvIII), AGS-15E (anti-SLTRK6), BAY1129980 (anti-C4.4a), BAY1187982 (anti-FGFR2), anetumab ravtansine (anti-mesothelin), GSK2857916 (anti-BCMA), tisotumab vedotin (anti-TF), IMGN289 (anti-EGFR), IMGN529 (anti-CD37), mirvetuximab soravtansine (anti-FOLR1), LOP628 (anti-c-KIT), PCA062 (anti-p-cadherin), BMS936561 (anti-CD70), MEDI-547 (anti-EphA2), PF-06263507 (anti-5T4), PF-06647020, PF-06647263 (anti-ephrin A), PF-06664178 (anti-TROP2), RG7450 (anti-STEAP1), RG7458 (anti-MUC16), RG7598, SAR566658 (anti-CA6), SGN-CD19A (anti-CD19), SGN-CD33A (anti-CD33), SGN-CD70A (anti-CD70), SGN-LIV1A (anti-LIV1), and trastuzumab vc-seco (anti-HER2).

Examples of ADCs comprising radionuclides include Y⁹⁰-ibritumomab tiuxetan and I¹³¹-tositumomab.

ADCs may be used to treat haematological cancers and solid tumours, for example, breast cancer, melanoma, lung cancer, SCLC, pancreatic cancer, colorectal cancer, ovarian cancer, endometrial cancer, cervical cancer, prostate cancer, mesothelioma, bladder cancer, RCC, liver carcinoma, gastric cancer, NSCLC, glioblastoma, head and neck cancer, oesophageal cancer, Hodgkin's lymphoma, NHL, anaplastic large cell lymphoma, ALL, DLBCL, multiple myeloma, CLL, and AML.

ADCs may be administered at a dose of 0.05 mg/kg to 16 mg/kg, for example.

Side effects of ADCs include thrombocytopenia, neutropenia, ocular toxicity, rash, typhlitis, nausea, dyspnea, liver toxicity, mucositis, anaemia, neuropathy, capillary leak syndrome, and diarrhoea.

Indications

Immunotherapy may be used to treat acute lymphoblastic leukaemia (ALL), acute myeloid leukaemia (AML), anaplastic large cell lymphoma, B cell lymphomas or leukaemias, B cell malignancies, bladder cancer, breast cancer, cervical cancer, chronic granulomatous disease, chronic lymphocytic leukaemia (CLL), chronic myeloid leukaemia (CML), colon cancer, colorectal cancer, diffuse large B cell lymphoma (DLBCL), endometrial cancer, Ewing's sarcoma, follicular lymphoma, gastric cancer, glioblastoma, glioma, haematological cancers, haematopoietic cancers, hairy cell leukaemia, head and neck cancer, head and neck squamous cell carcinoma, haematologic malignancy, hepatitis B/C, hepatoma, Hodgkin's lymphoma, Kaposi's sarcoma, liver carcinoma, lung cancer, lymphomas, malignant melanoma, malignant osteoporosis, malignant pleural mesothelioma (MPM), mantle cell lymphoma (MCL), Merkel cell carcinoma (MCC), melanoma, mesothelioma, multiple myeloma (MM), mycosis fungoides, neuroblastoma, neutropenia, non-Hodgkin's lymphoma (NHL), non-small cell lung carcinoma (NSCLC), metastatic melanoma, metastatic NSCLC, metastatic squamous NSCLC, oesophageal cancer, ovarian cancer, pancreatic cancer, primary mediastinal B cell lymphoma (PMBCL), renal cell carcinoma, sarcoma, small cell lung cancer (SCLC), solid tumours, T cell lymphoma, transformed follicular lymphoma (TFL), or urothelial cancer, for example.

Side Effects and Symptoms

Cytokine-release syndrome (CRS) is a serious side effect of immunotherapy. CRS is thought to result from proliferating T cells that release large quantities of cytokines, including IL-6, IFN-γ, TNF, IL-2, IL-2-receptor α, IL-8, IL-10, and GMCSF.

Symptoms of CRS include: high fever, malaise, fatigue, myalgia, nausea, anorexia, tachycardia/hypotension, capillary leak, cardiac dysfunction, renal impairment, hepatic failure, and disseminated intravascular coagulation.

Thus, subjects with CRS may experience any one or more of fever, cardiovascular symptoms including tachycardia, hypotension, arrhythmias, decreased cardiac ejection fraction, pulmonary symptoms including oedema, hypoxia, dyspnoea, and pneumonitis, acute renal injury usually caused by reduced renal perfusion, hepatic and gastrointestinal symptoms including elevated serum transaminases and bilirubin, diarrhoea, colitis, nausea, and abdominal pain, hematologic symptoms including cytopenias such as grade 3-4 anaemia, thrombocytopenia, leukopenia, neutropenia, and lymphopenia, derangements of coagulation including prolongation of the prothrombin time and activated partial thromboplastin time (PTT), D-dimer elevation, low fibrinogen, disseminated intravascular coagulation, macrophage activation syndrome (MAS), haemorrhage, B-cell aplasia, and hypogammaglobulinemia, infectious diseases including bacteremia, Salmonella, urinary tract infections, viral infections such as influenza, respiratory syncytial virus, and herpes zoster virus, musculoskeletal symptoms including elevated creatine kinase, myalgias and weakness, neurological symptoms including delirium, confusion, and seizure.

MAS overlaps clinically with CRS with subjects potentially experiencing hepatosplenomegaly, lymphadenopathy, pancytopenia, liver dysfunction, disseminated intravascular coagulation, hypofibrinogenemia, hyperferritinemia, and hypertriglyceridemia. Like CRS, subjects with MAS exhibit elevated levels of cytokines, including IFN-γ and GMCSF.

On-target, off-cancer effects may lead to outcomes similar to GVHD and B cell aplasia, which is caused when the target cancer antigen is expressed endogenously on other healthy/normal cells types.

For instance, B cell aplasia occurs because anti-CD19 antibodies also target normal B cells that express CD19. The consequence of B cell aplasia is a reduced capacity to fight infection because of hypoimmunoglobulinemia. Intravenous immunoglobulin replacement therapy is used to prevent infection.

Another side effect of immunotherapy is TLS, which occurs when the contents of cells are released as a result of therapy causing cell death, most often with lymphoma and leukaemia. TLS is characterised by blood ion and metabolite imbalance, and symptoms include nausea, vomiting, acute uric acid nephropathy, acute kidney failure, seizures, cardiac arrhythmias, and death.

Neurotoxicity may result from immunotherapy and symptoms may include cerebral oedema, delirium, hallucinations, dysphasia, akinetic mutism, headache, confusion, alterations in wakefulness, ataxia, apraxia, facial nerve palsy, tremor, dysmetria, and seizure.

Anaphylaxis can arise from non-host proteins, such as murine-derived proteins forming part of the immunotherapy, e.g. chimeric or humanised mAb.

Subjects undergoing immunotherapy as disclosed herein may experience one or more side effects or symptoms including anaemia, aphasia, arrhythmia, arthralgia, back pain, blood and bone marrow disorders, blood and lymphatic system disorders, cardiac disorders, chills, coagulation disorders, colitis, confused state, constitutional symptoms, cough, decreased appetite, diarrhoea, disorientation, dizziness, dyspnea, encephalopathy, fatigue, fever, gastrointestinal disorders, general cardiovascular disorders, haemorrhage, hepatic disorders, hyperglycaemia, hypokalaemia, hypothyroidism, increased ALT, increased AST, increased C-reactive protein, infection febrile neutropenia, leukopenia, malaise, abnormal metabolic laboratory-testing results, metabolism nutrition disorders, mucosal inflammation, musculoskeletal disorders, myalgia, nausea, nervous system disorders, neurologic disorders, neutropenia, oedema, pain, palmar-plantar erythrodysesthesia, paresthesia, pneumonia, pruritus, pulmonary disorders, pyrexia, rash, renal genitourinary disorders, respiratory disorders, skin and subcutaneous tissue disorders, somnolence, speech disorders, sweats, thoracic mediastinal disorders, thrombocytopenia, tremor, tumour flare, tumour lysis syndrome, vascular disorders, and vomiting.

In turn, any of the side effects or symptoms listed above may be indicative of CRS, MAS, TLS, on-target, off-cancer effects, neurotoxicity, or anaphylaxis, for instance.

Management of Side-Effects

In general, side-effects of immunotherapy are managed with standard supportive therapy for any presenting symptoms. However, given the myriad side effects and symptoms that may occur as a result of immunotherapy, multiple supportive therapies may be required simultaneously. The mainstay of supportive therapy is steroids, for example dexamethasone, although more recently anti-cytokine therapies have been used to treat CRS, for example etanercept, an anti-TNF molecule, and tocilizumab, an anti-IL-6 receptor antibody.

Recommendations have been made that upon presentation of a grade 3 or grade 4 adverse event, immunotherapy should be discontinued permanently.

Mesenchymal Stem Cells

Accordingly, the invention provides an improved therapy for reducing the number, severity and duration of side effects caused by immunotherapy, by administration of mesenchymal stem cells (MSCs). MSCs exert their effects through their immunomodulatory properties, so for many side effects and symptoms, MSCs are able to act directly at the immunogenic cause of the side effect or symptom.

MSCs secrete bioactive molecules such as cytokines, chemokines and growth factors and have the ability to modulate the immune system. MSCs have been shown to facilitate regeneration and effects on the immune system without relying upon engraftment. In other words, the MSCs themselves do not necessarily become incorporated into the host—rather, they exert their effects and are then eliminated within a short period of time. However, MSCs may be engrafted.

As used herein, “mesenchymal stem cell” or “MSC” refers to a particular type of stem cell that may be isolated from a wide range of tissues, including bone marrow, adipose tissue (fat), placenta and umbilical cord blood. Alternatively, MSCs may be produced from pluripotent stem cells (PSCs). MSCs are also known as “mesenchymal stromal cells”. Alternatively or additionally, MSC refers to an MSC as defined by the Mesenchymal and Tissue Stem Cell Committee of the International Society for Cellular Therapy: (1) MSCs must be plastic-adherent when maintained in standard culture conditions; (2) MSCs must express CD105, CD73 and CD90, and lack expression of CD45, CD34, CD14 or CD11b, CD79alpha or CD19 and HLA-DR surface molecules; (3) MSCs must differentiate to osteoblasts, adipocytes and chondroblasts in vitro.

Production of MSCs from PSCs is described in international patent application no. PCT/AU2017/050228 filed 14 Mar. 2017, which is incorporated in full by this cross-reference, and is described in Examples 1 and 2.

MSCs have been shown to exert immunomodulatory activities against T cells, B cells, dendritic cells, macrophages, and natural killer cells. While not wishing to be bound by theory, the underlying mechanisms may comprise immunomodulatory mediators, for example nitric oxide, indoleamine 2,3, dioxygenase, prostaglandin E2, tumour necrosis factor-inducible gene 6 protein, CCL-2, and PD-L1. These mediators are expressed at a low level until stimulated, for example by an inflammatory cytokines, such as IFNγ, TNF, and IL-17.

In one embodiment, MSCs are pre-treated prior to administration. Pre-treatment may be with a growth factor or by gene editing, for example, where a growth factor may prime the MSC and gene editing may confer a new or improved, e.g. more potent, therapeutic property on the MSC.

As used herein, “pluripotent stem cell” or “PSC” refers to a cell that has the ability to reproduce itself indefinitely, and to differentiate into any other cell type. There are two main types of PSC: embryonic stem cells (ESCs) and induced pluripotent stem cells (iPSCs).

As used herein, “embryonic stem cell” or “ESC” refers to a cell isolated from a five to seven day-old embryo donated with consent by subjects who have completed in vitro fertilisation therapy, and have surplus embryos. The use of ESCs has been hindered to some extent by ethical concerns about the extraction of cells from human embryos.

Suitable human PSCs include H1 and H9 human embryonic stem cells.

As used herein, “induced pluripotent stem cell” or “iPSC” refers to an ESC-like cell derived from adult cells. iPSCs have very similar characteristics to ESCs, but avoid the ethical concerns associated with ESCs, since iPSCs are not derived from embryos. Instead, iPSCs are typically derived from fully differentiated adult cells that have been “reprogrammed” back into a pluripotent state.

Suitable human iPSCs include, but are not limited to, iPSC 19-9-7T, MIRJT6i-mND1-4 and MIRJT7i-mND2-0 derived from fibroblasts and iPSC BM119-9 derived from bone marrow mononuclear cells. Other suitable iPSCs may be obtained from Cellular Dynamics International (CDI) of Madison, Wis., USA.

In one embodiment, MSCs used according to the invention are formed from primitive mesodermal cells. The primitive mesoderm cells may have mesenchymoangioblast (MCA) potential. The primitive mesoderm cells may have a ^{EMH}lin⁻KDR⁺APLNR⁺PDGFRalpha⁺ phenotype. In one embodiment, MSCs used according to the invention are formed from ^{EMH}lin⁻KDR⁺APLNR⁺PDGFRalpha⁺ primitive mesoderm cells with MCA potential.

A primitive mesoderm cell may be differentiated from a PSC, for example an iPSC, by culturing the PSC in a differentiation medium comprising FGF2, BMP4, Activin A, and LiCl under hypoxic conditions for about two days to form the primitive mesoderm cell.

Thus also disclosed is a method of differentiating a pluripotent stem cell (PSC) into a mesenchymal stem cell (MSC), the method comprising:
(a) culturing the PSC in a differentiation medium comprising FGF2, BMP4, Activin A, and LiCl under hypoxic conditions for about two days to form a primitive mesoderm cell;
(b) replacing the differentiation medium of (a) with a mesenchymal colony forming medium (M-CFM) comprising LiCl and FGF2, but excluding PDGF;
(c) culturing the primitive mesoderm cell of (b) in the M-CFM of (b) under normoxic conditions for sufficient time for a mesenchymal colony to form; and
(d) culturing the mesenchymal colony of (c) adherently to produce the MSC.

In some embodiments, the concentration in the differentiation medium of: BMP4 is about 10 ng/mL to about 250 mg/mL; FGF2 is about 5 ng/mL to about 50 ng/mL; activin A is about 1 ng/mL to about 15 ng/mL; and LiCl is about 1 mM to about 2 mM. In one embodiment, the differentiation medium comprises about 50 ng/mL BMP4; about 50 ng/mL FGF2; about 1.5 ng/mL activin A; and about 2 mM LiCl.

As used herein, “mesenchymoangioblast” and “MCA” refers to a common or bipotential mesenchymal cell and endothelial cell precursor.

As used herein, “^{EMH}lin⁻KDR⁺APLNR⁺PDGFRalpha⁺ primitive mesoderm cell with MCA potential” refers to a cell expressing typical primitive streak and lateral plate/extraembryonic mesoderm genes. These cells have potential to form MCA and hemangioblast colonies in serum-free medium in response to fibroblast growth factor 2 (FGF2). When cultured according to example 2, these cells become MSCs.

The term ^{EMH}lin⁻ denotes lack of expression of CD31, VE-cadherin endothelial markers, CD73 and CD105 mesenchymal/endothelial markers, and CD43 and CD45 hematopoietic markers.

In one embodiment, MSCs used according to the invention exhibit a CD73⁺CD105⁺CD90⁺CD146⁺CD44⁺CD10⁺CD31⁻CD45⁻ phenotype. Although not explicitly indicated, this phenotype conforms to the Mesenchymal and Tissue Stem Cell Committee of the International Society for Cellular Therapy definition of MSCs.

In one embodiment, MSCs used according to the invention express each of the microRNAs miR-145-5p, miR-181b-5p, and miR-214-3p, but not miR-127-3p and miR-299-5p.

MSCs possess “immunomodulatory activities”, which may be assessed in vitro as the capacity of a MSC to suppress proliferation of T helper (CD4⁺) lymphocytes. Immunomodulatory activities may be quantified in vitro relative to a reference, for example as determined using an ImmunoPotency Assay.

A suitable ImmunoPotency Assay uses an irradiated test MSC (e.g. iPSC-MSC produced according to the method disclosed herein) and an irradiated reference sample MSC, which are plated separately at various concentrations with carboxyfluorescein succinimidyl ester-labelled leukocytes purified from healthy donor peripheral blood. T helper (CD4⁺) lymphocytes that represent a subset of the reference sample are stimulated by adding CD3 and CD28 antibodies. CD4 labelled T cells are enumerated using flow cytometry to assess T cell proliferation. IC50 values are reported as a function of the reference sample. A higher IC50 value indicates a greater magnitude of suppression of proliferation of T helper (CD4⁺) lymphocytes and thus is indicative of superior T-cell immunomodulatory properties. MSC samples are irradiated prior to use in this assay to eliminate the confounding factor of their proliferative potential.

It will be appreciated by the person skilled in the art that the exact manner of administering to a subject a therapeutically effective amount of MSCs for treating a side effect or symptom of immunotherapy in a subject will be at the discretion of the medical practitioner. The mode of administration, including dose, combination with other agents, timing and frequency of administration, and the like, may be affected by the subject's condition and history.

The MSC may be administered as a therapeutic composition. As used herein, the term “therapeutic composition” refers to a composition comprising an MSC or population of MSCs as described herein that has been formulated for administration to a subject. The MSC may be formulated in and/or a therapeutic composition may comprise an excipient, carrier, buffer or other additive that facilitates administration of the MSC to a subject. Preferably, the therapeutic composition is sterile. In one embodiment, the therapeutic composition is pyrogen-free.

In one embodiment, an MSC of the disclosure, for example a MSC that expresses miR-145-5p, miR-181b-5p, and miR-214-3p, but not miR-127-3p and miR-299-5p, and/or has phenotype CD73⁺CD105⁺CD90⁺ CD146⁺CD44⁺CD10⁺CD31⁻CD45⁻, or a therapeutic composition of the disclosure is provided in a container, preferably a sterile container, preferably a pyrogen-free container. In one embodiment, the container is suitable for bolus administration, for example, a syringe. In another embodiment, the container is suitable for infusion, for example, an infusion bag.

The MSC will be formulated, dosed, and administered in a fashion consistent with good medical practice. Factors for consideration in this context include the particular type of disorder being treated and anticipated side effects or symptoms, the particular subject being treated, the clinical condition of the subject, the site of administration, the method of administration, the scheduling of administration, and other factors known to medical practitioners. The therapeutically effective amount of the MSC to be administered will be governed by such considerations.

Doses of MSCs may range from about 10³ cells/m² to about 10¹⁰ cells/m², for example about 10⁶ cells/m² to about 2×10⁸ cells/m², or about 10³ cells/m², about 5×10³ cells/n, about 10⁴ cells/m², about 5×10⁴ cells/m², about 10¹ cells/m², about 5×10⁵ cells/m², about 10⁶ cells/m², about 5×10⁶ cells/m², about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², or about 5×10¹⁰ cells/m².

Doses of MSCs may range from about 10³ cells/kg to about 10¹⁰ cells/kg, for example about 10⁶ cells/kg to about 2×10⁸ cells/kg, or about 10³ cells/kg, about 5×10³ cells/kg, about 10⁴ cells/kg, about 5×10⁴ cells/kg, about 10⁵ cells/kg, about 5×10⁵ cells/kg, about 10⁶ cells/kg, about 5×10⁶ cells/kg, about 10⁷ cells/kg, about 5×10⁷ cells/kg, about 10⁸ cells/kg, about 5×10⁸ cells/kg, about 10⁹ cells/kg, about 5×10⁹ cells/kg, about 10¹⁰ cells/kg, or about 5×10¹⁰ cells/kg.

Doses of MSCs may range from about 10³ cells to about 10¹⁰ cells, for example about 10⁶ cells to about 2×10⁸ cells, or about 10³ cells, about 5×10³ cells, about 10⁴ cells, about 5×10⁴ cells, about 10⁵ cells, about 5×10⁵ cells, about 10⁶ cells, about 5×10⁶ cells, about 10⁷ cells, about 5×10⁷ cells, about 10⁸ cells, about 5×10⁸ cells, about 10⁹ cells, about 5×10⁹ cells, about 10¹⁰ cells, or about 5×10¹⁰ cells.

The MSCs may be administered in a single dose, a split dose, or in multiple doses. The MSCs may be administered more than once in a treatment cycle.

The MSCs may be administered to a subject systemically or peripherally by any suitable method, for example by routes including intravenous (IV), intra-arterial, intramuscular, intraperitoneal, intracerobrospinal, intracranial, subcutaneous (SC), intra-articular, intrasynovial, intrathecal, intracoronary, transendocardial, surgical implantation, topical and inhalation (e.g. intrapulmonary) routes. Most preferably, the MSCs are administered IV. MSCs may be administered in combination with a scaffold of biocompatible material.

The MSCs may be administered to the subject before, during or after immunotherapy. In one embodiment, MSCs are administered during inflammation. Accordingly, in one embodiment, MSCs are administered after immunotherapy has started, optionally after inflammation has commenced and/or pro-inflammatory cytokine release has commenced or increased relative to a control, for example relative to pre-administration of the immunotherapy. Without wishing to be bound by theory, it is thought that most benefit will be gained by administering the MSCs after immunotherapy. This is because immunotherapy is intrinsically an immune/inflammatory response, whereas MSCs exert immunomodulatory and anti-inflammatory effects. Thus, administering MSCs before, during or too early after immunotherapy may dampen the effect of the immunotherapy. Despite this theory, the invention is not restricted to such.

However, this apparent paradox is understood and accepted by those skilled in the art. For example, a primary treatment for CRS caused by immunotherapy is administration of steroids, which are profoundly immunosuppressive. Advantages of MSCs, for example, may include local immunomodulation versus systemic immunosuppression by steroids, lack of persistence in the body, providing a further line of defence in subjects who fail to respond to steroids or other immunosuppressive therapies, reduced toxicity and increased specificity versus steroids, and self-regulation by MSCs versus steroids. Reduced toxicity, increased specificity, and self-regulation are related, and by self-regulation, it is meant that MSCs are thought to have a capacity to reduce their immunomodulatory activity as the immune response of the side effect or symptom of immunotherapy dissipates, whereas steroids for example must be withdrawn by the physician, with an ensuing period of half-lives before the steroid concentration drops below the therapeutic concentration.

In one embodiment, treating comprises administering the MSC(s) within 24 hours after observing a side effect of immunotherapy. In another embodiment, the MSCs may be administered to the subject receiving immunotherapy about 7 days, about 6 days, about 5 days, about 4 days, about 72 hours, about 48 hours, about 36 hours, about 24 hours, about 16 hours, about 12 hours, about 8 hours, about 4 hours, about 3 hours, about 2 hours, about 60 min, about 45 min, about 30 min, about 15 min, or about 5 min after observing a side effect of immunotherapy.

Accordingly, in one embodiment, the MSCs may be administered to the subject receiving immunotherapy about 1 week after immunotherapy. In another embodiment, the MSCs may be administered to the subject receiving immunotherapy about 5 min after immunotherapy. In another embodiment, the MSCs may be administered to the subject receiving immunotherapy about 6 days, about 5 days, about 4 days, about 72 hours, about 48 hours, about 36 hours, about 24 hours, about 16 hours, about 12 hours, about 8 hours, about 4 hours, about 2 hours, about 60 min, about 45 min, about 30 min, about 15 min, or about 5 min after immunotherapy. In one embodiment, the MSCs may be administered to the subject receiving immunotherapy within about 24 hours to about 72 hours after immunotherapy.

In one embodiment, the MSCs may be administered to the subject receiving immunotherapy about 1 week before immunotherapy. In another embodiment, the MSCs may be administered to the subject receiving immunotherapy about 5 min before immunotherapy. In another embodiment, the MSCs may be administered to the subject receiving immunotherapy about 6 days, about 5 days, about 4 days, about 72 hours, about 48 hours, about 36 hours, about 24 hours, about 16 hours, about 12 hours, about 8 hours, about 4 hours, about 2 hours, about 60 min, about 45 min, about 30 min, or about 15 min before immunotherapy.

In another embodiment, the MSCs may be administered to the subject receiving immunotherapy at about the same time as or during immunotherapy.

As used herein, “immunotherapy” when used in the context of “before”, “during”, “undergoing”, “after”, “undergone” and similar means before, during, undergoing, after, or undergone administration of the immunotherapeutic agent, for example a checkpoint inhibitor, a bispecific T cell engager, a stimulator of interferon genes agonist, a RIG I like receptor agonist, a Toll-like receptor agonist, a cytokine, an antibody-cytokine fusion protein, or an antibody-drug conjugate. As used herein, “undergoing” includes subjects who will undergo immunotherapy, but are yet to be administered the immunotherapy.

The term “therapeutically effective amount” refers to an amount of MSCs effective to treat a side effect or symptom of immunotherapy in a subject.

The terms “treat”, “treating” or “treatment” refer to both therapeutic treatment and prophylactic or preventative measures, wherein the aim is to prevent, reduce, or ameliorate a side effect or symptom of immunotherapy in a subject or slow down (lessen) progression of a side effect or symptom of immunotherapy in a subject. Subjects in need of treatment include those already with the side effect or symptom of immunotherapy as well as those in which the side effect or symptom of immunotherapy is to be prevented or ameliorated.

The terms “preventing”, “prevention”, “preventative” or “prophylactic” refers to keeping from occurring, or to hinder, defend from, or protect from the occurrence of a side effect or symptom of immunotherapy. A subject in need of prevention may be prone to develop the side effect or symptom of immunotherapy.

The term “ameliorate” or “amelioration” refers to a decrease, reduction or elimination of a side effect or symptom of immunotherapy.

A side effect or symptom of immunotherapy may be quantified. A side effect or symptom of immunotherapy may be quantified on a semi-quantitative scale, for example 0 to 5, where 0 represents absence, 1 to 4 represent identifiable increases in severity, and 5 represents maximum severity. Clinical trials often use a 1 to 5 scale where: 1 represents a mild adverse event (side effect); 2 represents a moderate adverse event (side effect); 3 represents a severe adverse event (side effect); 4 represents a life-threatening or disabling adverse event (side effect); and 5 represents death related to adverse event (side effect). Alternatively, a side effect or symptom of immunotherapy may be quantified as a binary event, i.e. presence or absence, 0 or 1. Other semi-quantitative scales will be readily apparent to the person skilled in the art. In another embodiment, a side effect or symptom of immunotherapy may be quantified on a quantitative scale, for instance: mass per volume such as mass of cytokine per volume of tissue fluid; temperature; duration; rate; enzyme activity; oxygen saturation; and so on.

The person skilled in the art will readily understand how to assess and quantify any side effect or symptom of immunotherapy, and be able to do so without difficulty or undue burden. For example, the person skilled in the art will be able to measure: a cytokine concentration in plasma or serum; temperature (fever); heart rate (tachycardia); blood pressure (hypotension); cardiac dysfunction; renal impairment; serum or plasma enzyme concentrations (hepatic function); and so on.

Any quantification of a side effect or symptom of immunotherapy may be compared to a control, for example a healthy control subject receiving neither immunotherapy nor MSCs, an affected control subject receiving immunotherapy, but not treated with MSCs, or a population.

Treating a side effect or symptom of immunotherapy by administering a MSC may be about a 1% decrease, about a 2% decrease, about a 3 decrease, about a 4 decrease, about a 5 decrease, about a 6% decrease, about a 7% decrease, about an 8% decrease, about a 9% decrease, about a 10% decrease, about a 20% decrease, about a 30% decrease, about a 40% decrease, about a 50% decrease, about a 60% decrease, about a 70% decrease, about an 80% decrease, about a 90 decrease, about a 100r, or greater decrease in the side effect or symptom of immunotherapy. Alternatively, treating a side effect or symptom of immunotherapy may be about a 2-fold, about a 3-fold, about a 4-fold, about a 5-fold, about a 6-fold, about a 7-fold, about an 8-fold, about a 9-fold, about a 10-fold, or more decrease in the side effect or symptom of immunotherapy. It follows that “less severe side effects” refers to such a decrease in the side effect or symptom of immunotherapy.

As used herein, the term “subject” may refer to a mammal. The mammal may be a primate, particularly a human, or may be a domestic, zoo, or companion animal. Although it is particularly contemplated that the method disclosed herein is suitable for medical treatment of humans, it is also applicable to veterinary treatment, including treatment of domestic animals such as horses, cattle and sheep, companion animals such as dogs and cats, or zoo animals such as felids, canids, bovids and ungulates.

The following examples assist in describing the invention, which is not to be limited to such examples.

### EXAMPLES

TABLE 1
Reagents

| Description | Vendor/Cat # or Ref # |
|---|---|
| DMEM/F12 Base Medium | Invitrogen/A1516901 |
| E8 supplement | Invitrogen/A1517101 |
| vitronectin | Life Technologies/A14700 |
| collagen IV | Sigma/C5533 |
| H-1152 ROCK Inhibitor | EMD Millipore/555550 |
| Y27632 dihydrochloride ROCK | Tocris/1254 |
| Inhibitor | |
| FGF2 | Waisman Biomanufacturing/ |
| | WC-FGF2-FP |
| human endothelial-SFM | Life Technologies/11111-044 |
| stemline II hematopoietic | Sigma/S0192 |
| stem cell expansion medium | |
| GLUTAMAX | Invitrogen/35050-061 |
| insulin | Sigma/I9278 |
| lithium chloride (LiCl) | Sigma/L4408 |
| collagen I solution | Sigma/C2249 |
| fibronectin | Life Technologies/33016-015 |
| DMEM/F12 | Invitrogen/11330032 |
| recombinant human BMP4 | Peprotech/120-05ET |
| activin A | Peprotech/120-14E |
| Iscove's modified | Invitrogen/12200036 |
| Dulbecco's medium (IMDM) | |
| Ham's F12 nutrient mix | Invitrogen/21700075 |
| sodium bicarbonate | Sigma/S5761 |
| L-ascorbic acid 2-phosphate Mg²⁺ | Sigma/A8960 |
| 1-thioglycerol | Sigma/M6145 |
| sodium selenite | Sigma/S5261 |
| non-essential amino acids | HyClone/SH30853.01 |
| chemically defined lipid | Invitrogen/11905031 |
| concentrate | |
| embryo transfer grade water | Sigma/W1503 |
| polyvinyl alcohol (PVA) | MP Bio/151-941-83 |
| holo-transferrin | Sigma/T0665 |
| ES-CULT M3120 | Stem Cell Technologies/03120 |
| STEMSPAN serum-free expansion | Stem Cell Technologies/09650 |
| medium (SFEM) | |
| L-ascorbic acid | Sigma/A4544 |
| Platelet-derived growth factor | Peprotech/110-14B |
| subunit B homodimer (PDGF-BB) | |

### Example 1. Reagents for MSC Production

The reagents listed in Table 1 are known to the person skilled in the art and have accepted compositions, for example IMDM and Ham's F12. GLUTAMAX comprises L-alanyl-L-glutamine dipeptide, usually supplied at 200 mM in 0.85% NaCl. GLUTAMAX releases L-glutamine upon cleavage of the dipeptide bond by the cells being cultured. Chemically defined lipid concentrate comprises arachidonic acid 2 mg/L, cholesterol 220 mg/L, DL-alpha-tocopherol acetate 70 mg/L, linoleic acid 10 mg/L, linolenic acid 10 mg/L, myristic acid 10 mg/L, oleic acid 10 mg/L, palmitic acid 10 mg/L, palmitoleic acid 10 mg/L, pluronic F-68 90 g/L, stearic acid 10 mg/L, TWEEN 80*2.2 g/L, and ethyl alcohol. H-1152 and Y27632 are highly potent, cell-permeable, selective ROCK (Rho-associated coiled coil forming protein serine/threonine kinase) inhibitors.

TABLE 2
IF6S medium (10X concentration)

| | | | Final | |
|---|---|---|---|---|
| 10X IF6S | Quantity | | Concentration | |
| IMDM | 1 package, | | 5X | |
| | powder for 1 L | | | |
| Ham's F12 nutrient mix | 1 package, | | 5X | |
| | powder for 1 L | | | |
| sodium bicarbonate | 4.2 | g | 21 | mg/mL |
| L-ascorbic acid 2-phosphate Mg²⁺ | 128 | mg | 640 | μg/mL |
| 1-thioglycerol | 80 | μL | 4.6 | mM |
| sodium selenite (0.7 mg/mL solution) | 24 | μL | 84 | ng/mL |

| | | | |
|---|---|---|---|
| GLUTAMAX | 20 | mL | 10X |
| non-essential amino acids | 20 | mL | 10X |
| chemically defined lipid concentrate | 4 | mL | 10X |

| | | |
|---|---|---|
| embryo transfer grade water | To 200 mL | NA |

TABLE 3
IF9S medium (IX concentration; based on IF6S)

| | | | Final | |
|---|---|---|---|---|
| IF9S | Quantity | | Concentration | |
| IF6S | 5 | mL | 1X | |
| polyvinyl alcohol (PVA; 20 mg/mL | 25 | mL | 10 | mg/mL |
| solution) | | | | |
| holo-transferrin (10.6 mg/mL | 50 | μL | 10.6 | μg/mL |
| solution) | | | | |
| insulin | 100 | μL | 20 | μg/mL |

| | | | |
|---|---|---|---|
| embryo transfer grade water | To 50 | mL | NA |

TABLE 4
Differentiation medium (1X concentration; based on IF9S)

| | | |
|---|---|---|
| | | Final |
| Differentiation Medium | Quantity | Concentration |

| IF9S | 36 | mL | 1X | |
|---|---|---|---|---|
| FGF2 | 1.8 | μg | 50 | ng/mL |
| LiCl (2M solution) | 36 | μL | 2 | mM |
| BMP4 (100 μg/mL solution) | 18 | μL | 50 | ng/mL |
| Activin A (10 mg/mL solution) | 5.4 | μL | 1.5 | ng/mL |

TABLE 5
Mesenchymal colony forming medium (1X concentration)

| | | |
|---|---|---|
| Mesenchymal colony forming | | Final |
| medium (M-CFM) | Quantity | Concentration |

| ES-CULT M3120 | 40 | mL | 40% | |
|---|---|---|---|---|
| STEMSPAN SFEM | 30 | mL | 30% | |
| human endothelial-SFM | 30 | mL | 30% | |
| GLUTAMAX | 1 | mL | 1X | |
| L-ascorbic acid (250 mM solution) | 100 | μL | 250 | μM |
| LiCl (2M solution) | 50 | μL | 1 | mM |
| 1-thioglycerol (100 mM solution) | 100 | μL | 100 | μM |
| FGF2 | 600 | ng | 20 | ng/mL |

TABLE 6
Mesenchymal serum-free expansion medium (1X concentration)

| Mesenchymal serum-free expansion | | | Final | |
|---|---|---|---|---|
| medium (M-SFEM) | Quantity | | Concentration | |
| human endothelial-SFM | 5 | L | 50% | |
| STEMLINE II HSFM | 5 | L | 50% | |
| GLUTAMAX | 100 | mL | 1X | |
| 1-thioglycerol | 87 | μL | 100 | μM |
| FGF2 | 100 | μg | 10 | ng/mL |

### Example 2. Protocol for Differentiating Human PSC into MSC

1. Thawed iPSCs in E8 Complete Medium (DMEM/F12 Base Medium+E8 Supplement)+1 μM H1152 on Vitronectin coated (0.5 μg/ckg) plastic ware. Incubated plated iPSCs at 37° C., 5% CO₂, 20% O₂ (normoxic).
2. Expanded iPSCs three passages in E8 Complete Medium (without ROCK inhibitor) on Vitronectin coated (0.5 μg/ckg) plastic ware and incubated at 37° C., 5% CO₂, 20% O₂ (normoxic) prior to initiating differentiation process.
3. Harvested and seeded iPSCs as single cells/small colonies at 5×10³ cells/ckg on Collagen IV coated (0.5 μg/ckg) plastic ware in E8 Complete Medium+10 μM Y27632 and incubated at 37° C., 5% CO₂, 20% O₂ (normoxic) for 24 h.
4. Replaced E8 Complete Medium+10 μM Y27632 with Differentiation Medium and incubated at 37° C., 5% CO₂, 5% O₂ (hypoxic) for 48 h to produce primitive mesoderm cells.
5. Harvested colony forming primitive mesoderm cells from Differentiation Medium adherent culture as a single cell suspension, transferred to M-CFM suspension culture and incubated at 37° C., 5% CO₂, 20% O₂ (normoxic) for 12 days, until mesenchymal colonies formed.
6. Harvested and seeded mesenchymal colonies on Fibronectin/Collagen I coated (0.67 μg/ckg Fibronectin, 1.2 μg/ckg Collagen I) plastic ware in M-SFEM and incubated at 37° C., 5% CO₂, 20% O₂ (normoxic) for 3 days to produce MSCs (Passage 0).
7. Harvested colonies and seeded as single cells (Passage 1) at 1.3×10⁴ cells/ckg on Fibronectin/Collagen 1 coated plastic ware in M-SFEM and incubated at 37° C., 5% CO₂, 20% O₂ (normoxic) for 3 days.
8. Harvested and seeded as single cells (Passage 2) at 1.3×10⁴ cells/ckg on Fibronectin/Collagen 1 coated plastic ware in M-SFEM and incubated at 37° C., 5% CO₂, 20% O₂ (normoxic) for 3 days.
9. Harvested and seeded as single cells (Passage 3) at 1.3×10⁴ cells/ckg on Fibronectin/Collagen 1 coated plastic ware in M-SFEM and incubated at 37° C., 5% CO₂, 20% O₂ (normoxic) for 3 days.
10. Harvested and seeded as single cells (Passage 4) at 1.3×10⁴ cells/ckg on Fibronectin/Collagen 1 coated plastic ware in M-SFEM and incubated at 37° C., 5% CO₂, 20% O₂ (normoxic) for 3 days.
11. Harvested and seeded as single cells (Passage 5) at 1.3×10⁴ cells/ckg on Fibronectin/Collagen 1 coated plastic ware in M-SFEM and incubated at 37° C., 5% CO₂, 20% O₂ (normoxic) for 3 days.
12. Harvested as single cells and froze final product.

Two experiments (TC-A-96 and DAD-V-90) were executed to investigate the impact of supplementing M-CFM with PDGF-BB (10 ng/mL) and/or LiCl (1 mM) on T cell suppression of iPSC-MSCs. T cell suppression was evaluated generated using Waisman Biomanufacturing's ImmunoPotency Assay (IPA).

As outlined in Table 7, the following combinations of platelet-derived growth factor (PDGF) and LiCl were evaluated: PDGF+/LiCl+, PDGF−/LiCl−, PDGF+/LiCl− and PDGF−/LiCl+. Note that two different Dneg1 seed densities (5×10³ cells/ckg and 1×10⁴ cells/ckg) and two different concentrations of activin A (AA) in the Differentiation Medium (1×AA=15 ng/mL and 0.1×AA=1.5 ng/mL) were compared in the TC-A-96 experiment. A single Dneg1 seed density (5×10³ cells/ckg) and activin A concentration (1.5 ng/mL) were used in the DAD-V-90 experiment. Also note that a single leukopak (LPK7) was used in the first IPA (IPA 1) and two leukopaks (LPK7 and LPK8) were used in the second IPA (IPA 2).

This assay is designed to assess the degree to which each MSC line can suppress the proliferation of T helper (CD4⁺) lymphocytes. Cryopreserved MSCs were tested using cryopreserved leukocytes purified from the peripheral blood of healthy individuals (peripheral blood mononucleocyte cells (PBMC) derived from Leucopaks (LPK)). As such, LPK cell population variation is expected from donor to donor. Each MSC test sample was tested against the PMBC from two different individuals for clinical grade material with the option to limit testing to a single PMBC sample for research grade material. The assay for each MSC test sample was run in conjunction with a reference standard MSC line to ensure assay integrity/reproducibility and to normalize test samples. The assay is described in Bloom et al. Cytotherapy, 2015, 17(2):140-51.

In brief, test MSCs were exposed to 21 Gy of gamma irradiation. In a 48-well tissue culture plate 4×10⁵, 2×10⁵, 4×10⁴, and 2×10⁴ irradiated MSCs were plated into individual wells. PMBC were separately labelled with carboxyfluorescein succinimidyl ester. Labelled PMBC cells are plated at 4×10⁵ cells per well containing the MSCs above. This results in titrated PBMC:MSC ratios of 1:1, 1:0.5, 1:0.1, and 1:0.05. An additional well was plated with stimulated PBMCs alone, another with MSCs alone, and another 1:0.05 ratio without stimulation, all which serve as controls. Subsequently, T cell-stimulatory monoclonal antibodies, anti-human CD3-epilson and anti-human CD28 (R&D Systems, Inc., Minneapolis, Minn.), were added to each well.

On day four of culture, cells were harvested from individual wells. Cells from each well were incubated with allophycocyanin-labelled anti-human CD4. CD4⁺ cells were then analysed for proliferation via carboxyfluorescein intensity using a flow cytometer. The MSC alone control served to gate out MSCs from co-culture wells. The PBMC alone control served as the positive control for maximum T cell proliferation against which the degree of MSC mediated suppression is measured. The non-stimulated 1:0.05 ratio well was used to generate a negative control gate against which proliferation was measured.

From test sample ratios a best fit curve was used to generate IC50 values. The IC50 values were normalized to the reference standard (IC50 Ref Std/IC50 Test Sample). This normalized IC50 yields larger values for more potent (more suppressive) samples and smaller values for less potent samples.

Results

IC50 data presented in Table 7 show that M-CFM supplemented with LiCl, but excluding PDGF (i.e. PDGF−/LiCl+) was optimal for differentiating iPSCs to produce iPSC-MSCs that are immunomodulatory. Furthermore, a lower concentration of activin A also improved the immunosuppression of iPSC-MSCs.

TABLE 7
ImmunoPotency Assay

| IC50 | IC50 | | | | | | |
|---|---|---|---|---|---|---|---|
| (LPK7) | (LPK8) | Sample | PDGF | LiCl | Activin A | | Seed Density (D2) |
| NA | NS | TC-A-96-B3 | + | + | 0.1× | (1.5 ng/mL) | 5 × 10³ cells/cm² |
| NA | 0.17 | TC-A-96-B1 | + | + | 1× | (15 ng/mL) | 5 × 10³ cells/cm² |
| NA | 0.17 | DAD-V-90-4 | + | + | 0.1× | (1.5 ng/mL) | 5 × 10³ cells/cm² |
| NA | 0.19 | TC-A-96-D3 | + | + | 0.1× | (1.5 ng/mL) | 1 × 10⁴ cells/cm² |
| NA | 0.36 | DAD-V-90-2 | + | − | 0.1× | (1.5 ng/mL) | 5 × 10³ cells/cm² |
| NA | 0.57 | DAD-V-90-1 | − | − | 0.1× | (1.5 ng/mL) | 5 × 10³ cells/cm² |
| 0.39 | 0.54 | TC-A-96-B2 | − | + | 1× | (15 ng/mL) | 5 × 10³ cells/cm² |
| 0.37 | 0.58 | TC-A-96-D2 | − | + | 1× | (15 ng/mL) | 1 × 10⁴ cells/cm² |
| 0.69 | 0.93 | DAD-V-90-3 | − | + | 0.1× | (1.5 ng/mL) | 5 × 10³ cells/cm² |

NA not applicable,
NS not suppressive
MSCs produced according to this example exhibit a CD73⁺CD105⁺CD90⁺CD146⁺CD44⁺CD10⁺CD31⁻CD45⁻ phenotype.

### Example 3. MSC microFMA Analysis

The MSC produced according to Example 2 underwent analysis against a microRNA (miRNA) microarray comprising 1194 miRNAs and a proprietary miRNA panel consisting of miR-127-3p, miR-145-5p, miR-181b-5p, miR-214-3p, miR-299-5p, validated against 71 MSC samples and 94 non-MSC samples.

The MSC produced according to Example 2 expressed each of miR-145-5p, miR-181b-5p, and miR-214-3p, but not miR-127-3p and miR-299-5p.

### Example 4. Alternative Immunopotency Assay 1

Immunopotency of MSCs will be evaluated as follows: human PBMCs from various donors are pooled (to minimise inter-individual variability in immune response) in phosphate-buffered saline and stained with carboxyfluorescein succinimidyl ester (CFSE, 2 μM) for 15 minutes at 37° C. in the dark, at a cell density of 2×10⁷ PBMCs/mL. The reaction will be stopped by adding an equal amount of RPMI-1640 medium supplemented with 10% human blood group AB serum. 3×10⁵ CFSE labelled PBMCs resuspended in RPMI-1640 medium supplemented with 10% pooled human platelet lysate, 2 IU/mL preservative-free heparin (Biochrom), 2 mM L-glutamine, 10 mM (4-(2-hydroxyethyl)-1-piperazineethanesulfonic acid) (HEPES; Gibco), 100 IU/mL penicillin (Sigma) and 100 μg/mL streptomycin (Sigma) will be then plated per well in triplicate in 96-well flat-bottomed plates (Corning). T-cell proliferation will be determined using a Gallios 10-color flow cytometer and the Kaluza G1.0 software (both Coulter). Viable 7-aminoactinomycin-D-excluding (7-AAD−; BD Pharmingen) CD3⁻ APC⁺ (eBioscience) T cells will be analysed after 4 to 7 days. Proliferation kinetics and population distribution will be analysed using Modfit 4.1 software (Verity).

### Example 5. Alternative Immunopotency Assay 2

Immunopotency of MSCs will be evaluated as follows: T helper (CD4⁺) lymphocytes will be stained with CellTrace violet (CTV; Invitrogen) according to the manufacturer's instructions and then stimulated with anti-CD3/CD28-coated beads (Dynabeads, Invitrogen) at a T-cell/bead ratio of 5:1 in 96-well U-bottomed plates. Responder CD4 T cells will be then incubated with irradiated (at 100 Gy) Karpas 299 cells (K299 cells; Sigma) as a reference standard, or MSCs. The co-cultured cells will be incubated at 37° C. in 5% CO₂ in RPMI-1640 medium for 72 h. The cells will be then washed with AnnexinV binding buffer (BD Biosciences) and stained with Annexin V-fluorescein isothiocyanate or APC (BD Biosciences) for 15 min in the dark at room temperature. After this incubation, the cells will be stained with propidium iodide (PI) (Molecular Probes) and then data immediately acquired on a LSRII Fortessa (BD Biosciences). Collected data will be analysed with the use of FlowJo software (version 8.8.6; Tree Star). The viability is measured by the population of Annexin V-negative and PI-negative T cells. This proportion of viable cells will be analysed for CTV dim (% proliferation).

Suppression of T-cell proliferation will be calculated by means of the equation: % Suppression=100−(a/b*100), where a is the percentage proliferation in the presence of suppressor cells and b is the percentage proliferation in the absence of suppressor cells.

### Example 6. SCs Treat Side Effects of Checkpoint Inhibitor Therapy for Untreated Melanoma

Subjects with histologically confirmed stage III (unresectable) or stage IV melanoma and who have not received prior systemic treatment for advanced disease will be divided randomly into two groups. Each group will be infused IV with 3 mg/kg nivolumab every 2 weeks. Patients will be monitored intermittently for response (progression-free survival), and continuously for toxicity and side effects.

In one group of subjects, each subject will be infused IV with 1×10⁶ to 1×10⁷ MSCs per kg body weight within 24 hours of observing a side effect after infusing that subject with nivolumab.

In all subjects, progression-free survival is anticipated to be 6.9 months (anticipated 95% CI 4.3 to 9.5 months), an objective response rate is anticipated to be 44% (anticipated 95% CI 38 to 50%), and a complete response rate is anticipated to be 9%.

Treatment-related side effects of any grade are anticipated in 82% of subjects in the group receiving nivolumab alone, including diarrhoea (19% expected), fatigue (34% expected), pruritus (19% expected), rash (26% expected), nausea (13% expected), pyrexia (6% expected), decreased appetite (11% expected), increased ALT (4% expected), increased AST (4% expected), vomiting (6% expected), hypothyroidism (9% expected), colitis (1% expected), arthralgia (8% expected), and dyspnea (5% expected).

Subjects who are infused with MSCs are expected to exhibit less severe and possibly fewer side effects compared with subjects not treated with MSCs.

### Example 7. MSCs Treat Side Effects of Bispecific T-Cell Engager Therapy for Relapsed/Refractory DLBCL

Subjects with relapsed/refractory DLBCL will be divided randomly into two groups. Both groups will receive a stepwise dose of blinatumomab, with 9 mg/d in week 1, 28 mg/d in week 2, and 112 mg/d thereafter. Blinatumomab will be administered by continuous IV infusion for 8 weeks (cycle 1), 4 weeks without treatment, then treatment for 4 weeks (cycle 2).

Subjects will be monitored intermittently for response, and continuously for toxicity and side effects. Response will include overall response rate at week 10 after two cycles of blinatumomab, complete response rate, duration of response, progression-free survival, overall survival, and the incidence and severity of side effects.

Overall response rate will be determined by independent central radiologic assessment according to the Cheson revised response criteria for malignant lymphomas, including contrast-enhanced computed tomography at week 10 and fluorodeoxyglucose positron emission tomography (PET) examination at week 11.

In one group of subjects, each subject will be infused IV with 1×10⁶ to 1×10⁷ MSCs per kg body weight within 24 hours of observing a side effect after infusing that subject with blinatumomab.

A complete response is anticipated in 19% of subjects. The overall response rate amongst all subjects is anticipated to be 35%. Subjects with refractory disease at baseline are expected to have overall response rate of 19%. Subjects with relapsed disease at baseline are expected to have overall response rate of 67%. The median duration of response is expected to be 11.6 months overall.

In all subjects, median progression-free survival is anticipated to be 3.7 months, with a median follow-up of 15.0 months. In all subjects, median overall survival is anticipated to be 5.0 months, with a median follow-up of 11.7 months.

Anticipated side effects in subjects not administered MSCs include tremor (48% expected), pyrexia (44% expected), fatigue (26% expected), oedema (26% expected), thrombocytopenia (22% expected), pneumonia (22% expected), diarrhoea (22% expected), leukopenia (17% expected), increased C-reactive protein (17% expected), hyperglycaemia (17% expected), speech disorder (17% expected), cough (17% expected), back pain (17% expected), hypokalaemia (17% expected), dizziness (13% expected), encephalopathy (13% expected), aphasia (9% expected), somnolence (9% expected), disorientation (9% expected), confused state (9% expected), and paresthesia (9% expected).

Subjects who are infused with MSCs are expected to exhibit less severe and possibly fewer side effects compared with subjects not treated with MSCs.

### Example 9. MSCs Treat Side Effects of Antibody-Cytokine Fusion Therapy for Metastatic Breast Cancer

Subjects with metastatic breast cancer will be divided randomly into two groups. Both groups will receive IV F16-IL2 25 million international units (MIU) in combination with doxorubicin 25 mg/m². F16-IL2 is a human scFv specific for the A1 domain of tenascin-C, named F16, and fused to human cytokine IL2.

Each treatment cycle will comprise F16-IL2 and doxorubicin administration on days 1, 8 and 15 followed by 13 d rest. Tumour assessments will be performed according to Response Evaluation Criteria In Solid Tumours (RECIST).

Subjects will be monitored intermittently for response, and continuously for toxicity and side effects. Response will include objective response rate and progression-free survival.

In one group of subjects, each subject will be infused IV with 1×10⁶ to 1×10⁷ MSCs per kg body weight within 24 hours of observing a side effect after administering the F16-IL2 to that subject.

After 8 weeks (2 treatment cycles), the disease control rate is expected to be 67% in all subjects. In all subjects, median progression-free survival is expected to be 125 d and median overall survival is expected to be 351 d.

Anticipated side effects in subjects not administered MSCs include blood and lymphatic system disorders (at least 50% expected), cardiac disorders (7% expected), gastrointestinal disorders (at least 67% expected), metabolism and nutrition disorders (14% expected), nervous system disorders (7% expected), renal and urinary disorders (7% expected), respiratory, thoracic and mediastinal disorders (7% expected), skin and subcutaneous tissue disorders (at least 20% expected), and vascular disorders (7% expected).

Subjects who are infused with MSCs are expected to exhibit less severe and possibly fewer side effects compared with subjects not treated with MSCs.

### Example 9. MSCs Treat Side Effects of Cytokine Therapy for Advanced Melanoma

Subjects with stage IV or locally advanced stage III cutaneous melanoma and expressing of HLA*A0201 (to allow presentation of the peptide vaccine to T cells) will be assigned randomly to one of two groups. Once per cycle, subjects will receive 1 mg of gp100:209-217(210M) (amino acid sequence IMDQVPFSV) plus Freund's incomplete adjuvant (Montanide ISA-51) (the peptide vaccine) SC, followed by IL-2 (720 000 IU/kg) IV. IL-2 will be administered every 8 hours as bolus IV.

Each subject will be treated with IL-2, as tolerated, up to a maximum of 12 doses per cycle. Each cycle of treatment will be repeated every 3 weeks, with 1 extra week added after every two cycles to allow for evaluation of the response.

Subjects will be monitored intermittently for response, and continuously for toxicity and side effects. Response will include clinical response and progression-free survival.

In one group of subjects, each subject will be infused IV with 1×10⁶ to 1×10⁷ MSCs per kg body weight within 24 hours of observing a side effect after administering IL2 to that subject.

The response rate in both groups is anticipated to be 20%. Centrally verified overall clinical response in both groups is expected to be 16%. Progression-free survival in both groups is expected to be 2.2 months (expected 95% CI, 1.7 to 3.9 months). In both groups, the median overall survival is expected to be 17.8 months (expected 95% CI, 11.9 to 25.8 months).

Anticipated side effects in subjects not administered MSCs include blood or bone marrow (48% expected), general cardiovascular (36% expected), arrhythmia (19% expected), coagulation (4% expected), constitutional symptoms (28% expected), skin (7% expected), gastrointestinal (21% expected), haemorrhage (2% expected), hepatic (40% expected), infection or febrile neutropenia (8% expected), metabolic or laboratory-testing results (42% expected), musculoskeletal (7% expected), neurologic (26% expected), pulmonary (22% expected), pain (13% expected), renal or genitourinary (19% expected), and tumour lysis syndrome or tumour flare (2% expected).

Subjects who are infused with MSCs are expected to exhibit less severe and possibly fewer side effects compared with subjects not treated with MSCs.

### Example 10. MSCs Treat Side Effects of Antibody-Drug Conjugate Therapy for HER2-Positive Advanced Breast Cancer

Subjects with HER2-positive advanced (unresectable, locally advanced or metastatic) breast cancer will be divided randomly into two groups. Both groups will receive trastuzumab emtansine 3.6 mg/kg IV every 21 days.

Subjects will be monitored intermittently for response, and continuously for toxicity and side effects. Response will include progression-free survival, overall survival, and the objective response rate. Progression will be assessed according to modified RECIST.

In one group of subjects, each subject will be infused IV with 1×10⁶ to 1×10⁷ MSCs per kg body weight within 24 hours of observing a side effect after administering trastuzumab emtansine to that subject.

In both groups, median progression-free survival is expected to be 9.6 months, and median overall survival is expected to be 30.9 months. The objective response rate is expected to be 44%. The estimated 1 year survival rate is expected to be 85% and the estimated 2 year survival rate is expected to be 65%.

Anticipated side effects in subjects not administered MSCs include diarrhoea (23% expected), palmar-plantar erythrodysesthesia (1% expected), vomiting (19% expected), neutropenia (6% expected), hypokalaemia (9% expected), fatigue (35% expected), nausea (39% expected), mucosal inflammation (7% expected), anaemia (10% expected), elevated ALT (17% expected), elevated AST (22% expected), and thrombocytopenia (28% expected).

Subjects who are infused with MSCs are expected to exhibit less severe and possibly fewer side effects compared with subjects not treated with MSCs.

### Example 11. MSCs Treat Side Effects of TLR Agonist Therapy for Hematological Malignancy

Subjects with relapsed or refractory haematological malignancy will be divided randomly into two groups. Both groups will receive the TLR7 agonist 852A, a small-molecule imidazoquinoline, SC twice weekly for 12 weeks. Subjects will start dosing at 0.6 mg/m² twice weekly and escalate by 0.2 mg/m² after every 2 doses as tolerated to a target dose of 1.2 mg/m².

Subjects will be monitored intermittently for response, toxicity and continuously for side effects. Response will be assessed after every 8 doses (acute leukaemia) or every 12 doses (multiple myeloma or lymphomas). Response will include complete response, partial response, and stable disease.

In one group of subjects, each subject will be infused IV with 1×10⁶ to 1×10⁷ MSCs per kg body weight within 24 hours of observing a side effect after administering 852A to that subject.

The expected overall response rate in both groups is 18% at 1 year. The median survival in both groups is expected to be 3.5 months. A complete response of 6% is expected and a partial response of 6% is expected in both groups. Stable disease is expected in 12% of subjects in both groups.

Anticipated side effects in subjects not administered MSCs include nausea (88% expected), vomiting (18% expected), dyspnea (82% expected), fever (71% expected), chills (82% expected), myalgia (76% expected), sweats (94% expected), malaise (100% expected), oedema (59% expected), cough (47% expected), and pain (12% expected).

Subjects who are infused with MSCs are expected to exhibit less severe and possibly fewer side effects compared with subjects not treated with MSCs.

### Example 12. MSCs Treat Side Effects of RLR Agonist Therapy for Pancreatic Cancer

Subjects with pancreatic cancer will be divided randomly into two groups. Both groups will receive IV 2.9 mg/kg of a bifunctional ppp-siRNA that combines RIG-I activation with gene silencing of TGF-β₁ (ppp-TGF-β) twice weekly on days 1 and 4 over 6 weeks.

Subjects will be monitored intermittently for progression-free survival, and continuously for toxicity and side effects, which are expected to include increased ALT and leukopenia.

In one group of subjects, each subject will be infused IV with 1×10⁶ to 1×10⁷ MSCs per kg body weight within 24 hours of observing a side effect after administering ppp-TGF-β to that subject.

Subjects who are infused with MSCs are expected to exhibit less severe and possibly fewer side effects compared with subjects not treated with MSCs.

### Example 13. MSCs Treat Side Effects of STING Agonist Therapy for Colon Cancer

Subjects with colon cancer will be divided randomly into two groups. Both groups will receive IV 5 mg/kg of cGAMP twice weekly on days 1 and 4 over 6 weeks.

Subjects will be monitored intermittently for progression-free survival, and continuously for toxicity and side effects.

In one group of subjects, each subject will be infused IV with 1×10⁶ to 1×10⁷ MSCs per kg body weight within 24 hours of observing a side effect after administering cGAMP to that subject.

Subjects who are infused with MSCs are expected to exhibit less severe and possibly fewer side effects compared with subjects not treated with MSCs.

### Example 14. Prevention or Treatment of Cytokine Release Syndrome

This example uses NOD.Cg-Prkdc^{scid} Il2rg^{tm1Wjl}/SzJ (NSG) mice that are severely immunodeficient, which allows these mice to be humanized by engraftment and differentiation of peripheral blood mononuclear cells (PBMC) resulting in high percentages of human CD4+ and CD8+ T cells in the peripheral blood and the spleens of the mice. The OKT3 antibody binds to the human T cells and causes a strong induction of human cytokines, thereby modelling CRS in humans.

On day zero, 8- to 12-weeks-old female NOD.Cg-Prkdc^{scid} Il2rg^{tm1Wjl}/SzJ (NSG) mice were injected intravenously through the tail vein with 20×10⁶ human PBMC(huPBMC). A schematic representation of the experimental design is provided in FIG. 3.

Frozen human PBMC were purchased from StemCell Technologies and NSG mice were purchased from The Jackson Laboratory.

Frozen huPBMC samples were stored and thawed following the manufacturer's instructions. Briefly, the vial of frozen cells was thawed in a 37° C. water bath, the outside of the vial was cleaned with 70% ethanol, the cells were transferred to a 15 mL conical tube containing 10 mL of RPMI 10% FBS pre-warmed at 37° C., centrifuged at 1 500 rpm for 10 min, washed once with 10 mL of PBS and suspended in 1 mL of PBS for cell count by Trypan Blue dye exclusion. 20×10⁶ huPBMC aliquots in 150 μl of PBS were prepared and kept on ice while preparing the mice for injection.

Mice were placed in a cage and warmed for 2 to 3 minutes with a lamp to induce dilatation of the tail vein. Next, mice were placed in a mouse restrainer, the tails were cleaned with 70% ethanol, and mice were injected through the tail vein with 20×10⁶ huPBMC administered with a 1 ml syringe, 27G needle. After the injection, light pressure was applied to the site of the injection to prevent bleeding. Mice were monitored daily for signs of disease until the day of euthanasia.

10-12 days after huPBMC engraftment, mice were assigned to either a control (n=2) or test (n=5) cohort. Control cohorts received muromonab-CD3 (OKT3) antibody at a dose of 10 μg, via intraperitoneal injection. OKT3 antibody is an anti-CD3 antibody used as an immunosuppressant agent to treat acute rejection after organ transplant. OKT3 antibody may be purchased from commercial suppliers such as abcam (catalog no. ab86883) or ThermoFisher Scientific (catalog no. 14-0037-82) or other sources such as Walter and Eliza Hall Institute's Antibody Facility. The control and test cohort received OKT3 antibody via intraperitoneal injection 12 hours after huPBMC administration. The test cohort received 2×10⁶ MSCs by tail vein injection 5 hours before OKT3 administration (i.e. 7 hours after huPBMC administration). In other examples, MSCs will be administered at the same time as OKT3 administration or 1 h, 3 h, 5 h or 24 h after OKT3 administration (FIG. 3).

Temperatures of mice were acquired 0, 1, 3, 5, and 24 hours after administration of OKT3 antibody. Temperatures were taken using a non-contact, infrared thermometer that has been calibrated against a standard rectal thermometer to adjust for differences between rectal and surface/skin temperatures.

Five hours after administration of OKT3 antibody, peripheral blood samples were obtained via cheek vein puncture using a sterile 4 mm Goldenrod Animal Lancet or by withdrawing blood from the tail vein.

Mice were sacrificed 5 or 24 hours after OKT3 administration, depending on clinical score and body temperature. Peripheral blood samples were obtained immediately upon euthanasia via cardiac puncture, then spleens were harvested. Percent human CD45, CD4 and CD8 T cells found in circulation and in spleens was determined by standard flow cytometric staining and analysis. CD69 expression on circulating and splenic CD4 and CD8 T cells was assessed by surface staining and flow cytometric analysis.

Plasma samples collected at 5 and 24 hours after OKT3 administration will be evaluated for expression of IL-1β, IL-2, IL-6, IFNγ, TNF, IL-10, and optionally IL-4 and IL-5.

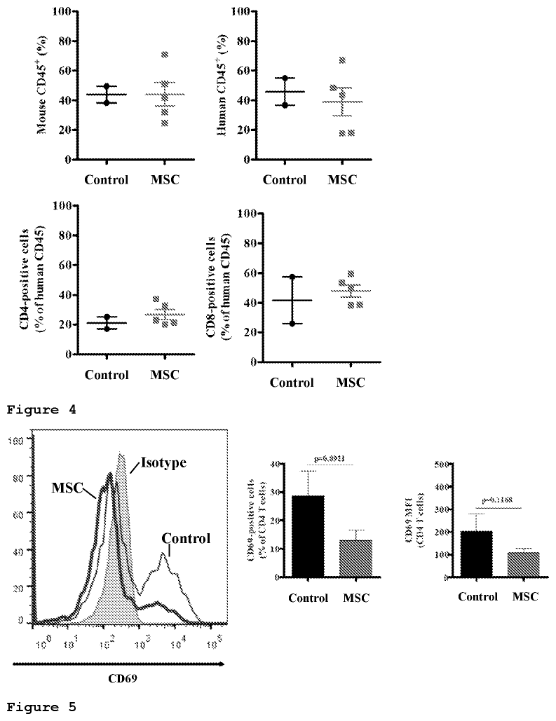

In this model of CRS, test mice exhibited a higher rectal temperature compared with control mice (FIG. 4). Also, test mice exhibited reduced clinical scores compared with test mice (FIG. 5) in this model of CRS.

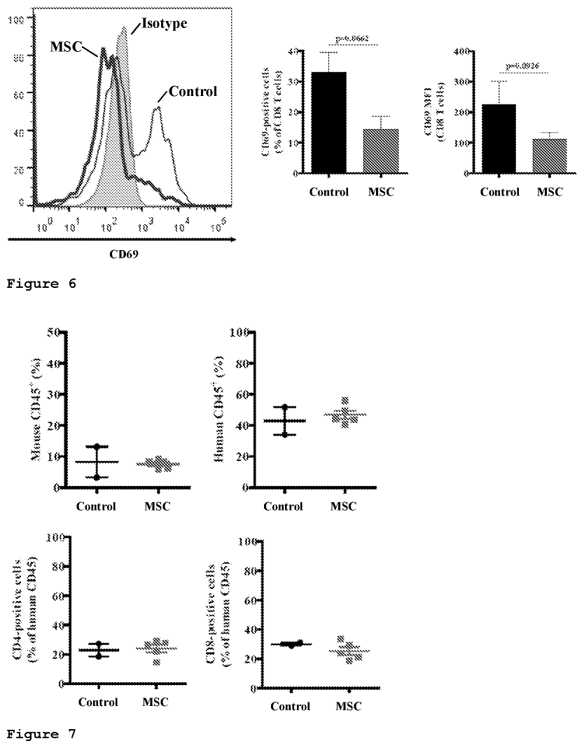

No difference was observed between control and test mice in the percentage of CD45+ cells in peripheral blood (FIG. 6) or spleen (FIG. 9).

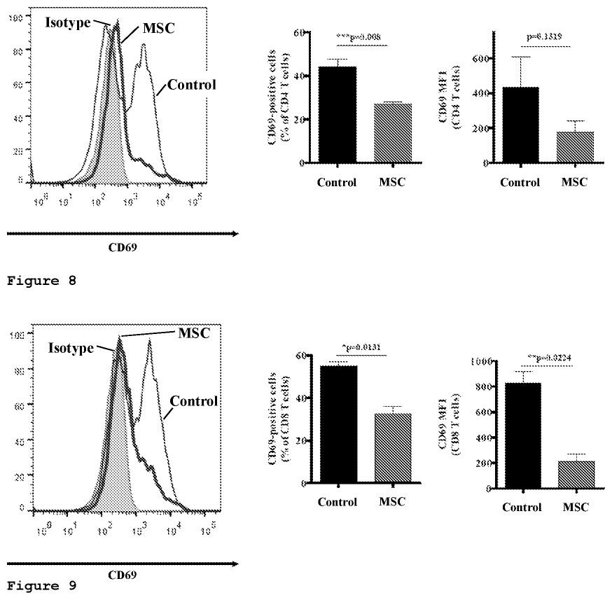

However, CD69 expression was reduced in test mice compared with control mice in human CD4+ cells in both peripheral blood (FIG. 7) and spleen (FIG. 10) and in human CD8+ cells in both peripheral blood (FIG. 8) and spleen (FIG. 11).

In view of reduced CD69 expression in human CD4+ cells and human CD8+ cells of test mice relative to control mice, expression of one or more of IL-1β, IL-2, IL-6, IFNγ, TNF, IL-10, IL-4 and IL-5 is expected to be reduced in test mice relative to control mice.

1. A method for treating a side effect of immunotherapy, the method comprising administering a mesenchymal stem cell (MSC) to a subject who has undergone or is undergoing immunotherapy, thereby treating a side effect of immunotherapy.

2. (canceled)

3. The method of claim 1, wherein the MSC has a CD73⁺CD105⁺CD90⁺CD146⁺CD44⁺CD10⁺CD31⁻CD45⁻ phenotype.

4. The method of claim 1, wherein the MSC expresses miR-145-5p, miR-181b-5p, and miR-214-3p, but not miR-127-3p and miR-299-5p.

5. The method of claim 1, wherein about 1×10⁶ MSCs/kg to about 1×10⁷ MSCs/kg are administered to the subject.

6. The method of claim 1, wherein the MSG MSC is administered to the subject before,
during or after immunotherapy.

7. The method of claim 1, wherein the MSC is administered to the subject after immunotherapy.

8. The method of claim 7, wherein the MSC is administered to the subject within 24 hours after observing a side effect of immunotherapy.

9. The method of claim 7, wherein the MSC is administered to the subject within 24 hours to 72 hours after immunotherapy.

10. The method of claim 1, wherein the side effect is cytokine release syndrome (CRS), optionally, release of interleukin-6 (IL-6), interferon-γ (IFN-7), tumour necrosis factor (TNF), IL-2, IL-2-receptor α, IL-8, IL-10, or granulocyte macrophage colony-stimulating factor (GMCSF); macrophage activation syndrome (MAS); an on-target, off-cancer effect, optionally, B cell aplasia; tumour lysis syndrome (TLS); neurotoxicity, optionally, cerebral edema; or anaphylaxis.

11. The method of claim 1, wherein the immunotherapy is for treating a lymphoma; a leukaemia; a melanoma; an epithelial cancer; or a sarcoma.

12. The method of claim 1, wherein the immunotherapy is for treating diffuse large B cell lymphoma (DLBCL); Hodgkin lymphoma; non-Hodgkin lymphoma (NHL); a non-Hodgkin B, T or NK cell lymphoma; primary mediastinal B cell lymphoma (PMBCL); transformed follicular lymphoma (TFL); mantle cell lymphoma (MCL); multiple myeloma (MM); chronic lymphocytic leukaemia (CLL); acute myeloid leukaemia (AML); or acute lymphoblastic leukaemia (ALL).

13. The method of claim 1, wherein the immunotherapy is a checkpoint inhibitor, a bispecific T cell engager, a stimulator of interferon genes agonist, a RIG I like receptor agonist, a Toll-like receptor agonist, a cytokine, an antibody-cytokine fusion protein, or an antibody-drug conjugate.

14. The method of claim 1, wherein the subject is mammalian, optionally human.

15. The method of claim 1, wherein the MSC is made by a method comprising:
(a) culturing a primitive mesoderm cell in a mesenchymal-colony forming medium (M-CFM) comprising LiCl and FGF2, but excluding PDGF, under normoxic conditions for sufficient time for a mesenchymal colony to form; and
(b) culturing the mesenchymal colony of =t(a) adherently to produce the MSC.

16.-17. (canceled)

18. The method of claim 1, wherein about 1×10⁶ MSCs to about 2×10⁸ MSCs are administered to the subject.

19. The method of claim 1, wherein about 1×10⁸ MSCs are administered to the subject.

20. The method of claim 1, wherein about 5×10⁸ MSCs are administered to the subject.
